(19) United States
(12) Patent Application Publication
(10) Pub. No.: US 2020/0256855 A1
(43) Pub. Date: Aug. 13, 2020

(54) METHODS AND DEVICES

(71) Applicant: MICROBIOSENSOR LIMITED, Manchester (GB)

(72) Inventors: Curtis Bryce DOBSON, Manchester (GB); Nishal GOVINDJI-BHATT, Manchester (GB); Duncan HENDERSON, Manchester (GB)

(21) Appl. No.: 16/651,760

(22) PCT Filed: Sep. 25, 2018
(86) PCT No.: PCT/GB2018/052725

§ 371 Date: Mar. 27, 2020

(30) Foreign Application Priority Data
Sep. 28, 2017 (GB) 1715704.1

(51) Int. Cl. G01N 33/50; C12Q 1/04

(52) U.S. Cl. CPC G01N 33/5094; G01N 2800/26; C12Q 1/04

(57) ABSTRACT

A method of determining the presence of a threshold concentration of leukocyte cells in a sample, the method comprising (i) contacting the sample with a leukocyte detection means comprising (a1) an indicator compound; and (ii) examining the leukocyte detection means to determine whether leukocytes are present.

The present invention relates to methods and devices for detecting leukocyte cells, and preferably also microorganisms, for example bacteria. Increased leukocyte cell concentration may be associated with infection and in particular with peritonitis.

Raised leukocyte levels (leukocytosis) is a sign of an inflammatory response. Increased leukocytes are often indicative of infection but can also occur due to non-infectious disease, injury, tumours or side-effects of medication.

Thus there are many occasions when a simple test which provides an early indication of raised leukocyte levels would be highly beneficial. Early detection can assist diagnosis and lead to quicker treatments and better patient outcomes.

In particular raised leukocyte levels can be an indication of microbial infection.

The rapid, reliable and accurate detection of infections is a vital part of both the treatment and prevention of infection and in particular in the treatment and prevention of infection of patients undergoing peritoneal dialysis.

It is particularly important to be able to detect infections in patients suffering from kidney failure. Patients with advanced chronic kidney failure can be treated by having two forms of renal replacement therapy (RRT) namely: peritoneal dialysis (PD) and haemodialysis (HD).

HD is the most commonly used RRT in the UK and many other countries despite the fact that PD is both more convenient for the patient (it can be done at home and gives patients the most freedom and flexibility) and requires fewer hospital visits and is less costly. However PD carries a greater risk of serious infection than HD, and often by the time infection becomes obvious, it can be life-threatening.

Currently patients rely on non-specific symptoms of pain & fever, and/or noticing that their dialysis effluent is cloudy, to alert them to infection. PD patients are commonly instructed to hold a page of text (a newspaper or the like) behind their PD effluent bag and if the text is obscured (i.e. by the effluent becoming cloudy) they are advised to contact their clinician. A problem with this approach is that the assessment is somewhat subjective. Furthermore PD fluid is likely to be clear when an infection is initially developing and it is often the case that by the time cloudiness is observed that the infection may be well developed and have become a serious risk to health.

It is an object of the present invention to provide a device and method which can be used to clearly alert a user, and at an earlier stage, that there are high levels of leukocytes in a sample.

According to a first aspect of the present invention there is provided a method of determining the presence of a threshold concentration of leukocyte cells in a sample, the method comprising (i) contacting the sample with a leukocyte detection means comprising (a1) an indicator compound; and (ii) examining the leukocyte detection means.

The present invention relates to a method of determining the presence of leukocyte cells in a sample. The sample may be any material in which such cells may be present. Suitably the sample is a sample of bodily fluid. Preferably the sample is selected from blood or components thereof, mucus, saliva, urine, pus, sputum, wound exudate, pleural fluid and fluid from the peritoneal cavity.

Preferably the sample is selected from blood or components thereof, mucus, saliva, urine, pus, sputum, wound exudate, pleural fluid and peritoneal dialysis effluent.

In some preferred embodiments the sample comprises fluid from the peritoneal cavity. Fluid from the peritoneal cavity can include fluid from peritoneal dialysis or fluid present in the abdomen due to a medical condition or disease (ascites).

Fluid may build up in the peritoneal cavity (ascites) due to a number of medical conditions or diseases including liver cirrhosis, cancer, heart failure, tuberculosis, pancreatitis, and blockage of the hepatic vein.

When there is fluid in the peritoneal cavity, there is a risk of peritonitis and early detection of infection is critical in being able to effectively treat such an infection.

Suitably the sample is selected from peritoneal dialysis effluent and fluid drained from the peritoneal cavity present due to ascites.

In especially preferred embodiments the sample is a peritoneal dialysis effluent. This may be referred to herein is PD effluent or PD fluid.

The indicator compound (a1) may be any compound that undergoes an observable change when a threshold concentration of leukocyte cells are present.

The observable change may be a change in light absorption, precipitate formation, bubble formation, temperature change or other measurable quality.

Preferably the observable change is a colour change. Suitably the indicator compound is a different colour in the presence of a threshold concentration of leukocyte cells than it is when no leukocyte cells or a low concentration of leukocyte cells are present. The indicator compound suitably has an initial colour before it is contacted with the sample. If the threshold concentration of leukocytes are present in the sample the indicator compound preferably undergoes a colour change. The indicator compound may change from colourless to coloured, from coloured to colourless, or from a first colour to a second colour which is different to the first colour.

The skilled person will appreciate that the colour change is suitably due to a change in the structure of the indicator compound which affects the chromophore region.

Preferably the indicator changes from colourless to coloured in the presence of a threshold concentration of leukocytes.

Reference to “activation” or “triggering” of the leukocyte detection means refers to a change in the leukocyte detection means, suitably a positive result indicating the presence of the threshold concentration of leukocytes.

The skilled person will appreciate that the threshold concentration will depend on the particular constituents of the leukocyte detection means and the amounts thereof. This will be taken into consideration by the skilled person when formulating the leukocyte detection means.

By threshold concentration we mean to refer to a level of leukocytes for which it is desired to provide a warning when this is reached i.e. an indication that the level is higher than expected/desirable. The threshold concentration may vary depending on the nature of the sample, the individual who provided the sample and the reason why the leukocyte level is being monitored.

Preferably the indicator compound is a colour change indicator. Preferably the indicator compound is a redox indicator.

Preferably the indicator compound (a1) undergoes a colour change when a threshold concentration of leukocyte cells in the test fluid is reached, i.e. the mixture obtained after admixture of the sample and the leukocyte detection means.

Preferably the threshold concentration of leukocyte cells is 10⁵ leukocyte cells/mL of test fluid.

A concentration of 10⁵ leukocyte cells/mL of dialysis fluid is an internationally recognised standard for the diagnosis of an infection in PD patients.

According to the International Society for Peritoneal Dialysis (ISPD); an effluent cell count with white blood cells (WBC) more than 100/μL (after a dwell time of at least 2 hours) indicates the presence of inflammation, with peritonitis being the most likely cause (Li et al., 2010. Peritoneal Dialysis-related Infections Recommendations: 2010 Update, Peritoneal Dialysis International, 30: 393-423).

Suitable indicator compounds for use in the leukocyte detection means include crystal violet, Carbol fuchsine, Safronin, Nigrosin, Indian ink, Iodine, Ziehl-Neelsen, Haemotoxylin, Eosin Y/Eosin yellowish, Papanicolaou, Orange G, Light green SF yellowish, Bismarck brown Y, Nile blue/Nile blue A, Nile red/Nile blue oxazone, Mason's trichome, Romanowsky, Wright's, Jenner's, Leishman, Giemsa, Silver, Sudan III, Sudan IV, Oil red O, Sudan Black B, Conklin, Malachite green, Osmium tetroxide/Tetraoxide, Rhodamine, Acridine Orange, Carmine, Coomassie blue, DAPI, Eosin B, Ethidium bromide, Acid fuchsine, Hoechst, Methylene green, Methylene blue, Neutral red/Toluylene red, and HDTMA/CTAB.

Examples of further indiactors that may be used include Resazurin (e.g. Alamar blue) and 10-acetyl-3,7-dihydroxyphenoxazine (Amplex Red). It is preferred that the indicator is activated by an enzyme endogenous to the leukocyte being detected and more preferred that the indicator is activated by the action of a cellular reductase (e.g. an NAD(P)H reductase).

The indicator compound is preferably a redox indicator. Suitably the indicator compound is reduced by the activity of cells in the sample.

Preferably the indicator compound used in the leukocyte detection means is a tetrazolium compound.

Preferred indicator compounds for use in the leukocyte detection means may include, for example, XTT (2,3-bis-(2-methoxy-4-nitro-5-sulfophenyI)-2H-tetrazolium-5-carboxanilide), MTS (3-(4,5-dimethylthiazol-2-yl)-5-(3-carboxymethoxyphenyl)-2-(4-sulfophenyl)-2H-tetrazolium), MTT (3-(4,5-Dimethylthiazol-2-yl)-2,5-diphenyltetrazolium bromide) or water soluble tetrazolium salts (WST) such as WST-1, WST-3, WST-4, WST-5, WST-7, WST-8, WST-9, WST-10 or WST-11. Alternatively, other tetrazolium salts may be used including indonitrotetrazolium chloride (INT), Nitrobluetetrazolium (NBT), Tetranitro blue tetrazolium (TNBT), Thiocarbamyl nitro blue tetrazolium (TCNBT), Tetrazolium red (TR), Tetrazolium Violet (TV), Neotetrazolium chloride (NTC) or 5-cyano-2,3-ditolyl tetrazolium chloride (CTC).

In one preferred embodiment the indicator compound (a1) is MTT (3-(4,5-Dimethylthiazol-2-yl)-2,5-diphenyltetrazolium bromide).

In another preferred embodiment the indicator compound (a) is WST-9.

The indicator compound (a1) (suitably a tetrazolium compound, for example MTT or WST-9) is preferably provided in an amount to provide a final concentration in the test fluid of at least 10 μg/mL fluid, preferably at least 50 μg/mL fluid, more preferably at least 100 μg/mL fluid, for example at least 190 μg/mL fluid.

The indicator compound (a1) (suitably a tetrazolium compound, for example MTT or WST-9) may be provided in an amount of up to 1000 μg/mL test fluid, suitably up to 750 μg/mL fluid, preferably up to 600 μg/mL, suitably up to 550 μg/mL, preferably up to 500 μg/mL fluid.

Preferably the leukocyte detection means further comprises (b) a buffer.

Preferably the buffer is selected to maintain the pH in the test fluid between 4 and 8, preferably between 5 and 7, more preferably between 6 and 6.5.

Any suitable buffer able to maintain pH within this range can be used. Suitable buffers will be known to the person skilled in the art and include, for example 2-(N-morpholino)ethanesulfonic acid (MES), 2,2-bis(hydroxymethyl)-2,2′,2″-nitrilotriethanol (BIS-TRIS), N-(2-acetamido)iminodiacetic acid (ADA), piperazine-N,N′-bis(2-ethanesulfonic acid) (PIPES), N-(2-acetamido)-2-aminoethanesulfonic acid (ACES), 3-morpholino-2-hydroxypropanesulfonic acid (MOPSO), 1,3-bis(tris(hydroxymethyl)methylamino)propane (BIS-TRIS Propane), N,N-bis(2-hydroxyethyl)-2-aminoethanesulfonic acid (BES), 3-morpholinopropane-1-sulfonic acid (MOPS), 2-[(2-hydroxy-1,1-bis(hydroxymethyl)ethyl)amino]ethanesulfonic acid (TES), 4-(2-hydroxyethyl)-1-piperazineethanesulfonic acid) (HEPES), 3-(N,N-bis[2-hydroxyethyl]amino)-2-hydroxypropanesulfonic acid (DIPSO), 2-hydroxy-3-[tris(hydroxymethyl)methylamino]-1-propanesulfonic acid (TAPSO), 2-amino-2-(hydroxymethyl)-1,3-propanediol (TRIZMA), piperazine-N,N′-bis(2-hydroxypropanesulfonic acid)/piperazine-1,4-bis(2-hydroxypropanesulfonic acid) dihydrate—hydrate (POPSO), 4-(2-hydroxyethyl)-1-piperazinepropanesulfonic acid (HEPPS), N-[tris(hydroxymethyl)methyl]glycine (TRICINE), diglycine (GLY-GLY), N,N-bis(2-hydroxyethyl)glycine (BICINE), N-(2-hydroxyethyl)piperazine-N′-(4-butanesulfonic acid) (HEPBS), N-[tris(hydroxymethyl)methyl]-3-aminopropanesulfonic acid (TAPS) and 2-amino-2-methyl-1,3-propandiol (AMPD).

One especially preferred buffer for use herein is MES (2-(N-morpholino)-ethane sulfonic acid).

In some embodiments activation of the indicator compound is optimised (with respect to threshold for activation and intensity of colour, for example) when an electron mediator is included in the reporting means that will promote the activity of redox enzyme systems.

In some embodiments the leukocyte detection means may further comprise (cl) an electron mediator.

Examples of electron mediators are well known to the art. For instance, the electron mediators listed by Fultz and Durst (Analytica Chimica Acta 140 (1992) 1-18).

Suitable electron mediators include viologens, phenzonium, phenothiazines, naphithanes, phenazines, indigos, indamine, indophenols, anthraquinones, naphthoquinones, benzoquinones and benzamines.

Preferably the electron mediator is selected from menadione or phenazine electron mediators.

Suitable phenazine electron mediators include N-methyl phenazine methosulphate (mPMS), phenazine methosulphate (PMS), phenazine ethosulphate (PES), pyocyanine, safranine O, safranine T, phenosafranine, benzophenazine and neutral red.

Preferred electron mediators are menadione, phenazine methosulphate and (PMS) derivatives thereof (e.g. phenazine ethosulphate). One especially preferred electron mediator for inclusion in the leukocyte detection means is 1-methoxy-5-methylphenazinium methylsulfate (mPMS).

The electron mediator, when present in an amount such that its final concentrations are greater than 0.001 mM in the tested fluid. For example it may be present in the range of 0.001-0.1 mM, and more preferably in the range 0.005-0.05 mM.

In some preferred embodiments the leukocyte detection means does not comprise an electron mediator.

In some preferred embodiments the leukocyte detection means does comprise an electron mediator.

Step (ii) of the method of the first aspect of the present invention involves examining the leukocyte detection means. The leukocyte detection means is suitably examined to determine whether leukocytes are present.

The skilled person will appreciate that this examination will be of the resultant composition that is obtained when the leukocyte detection means has been contacted with the sample, this may be referred to herein as “the tested fluid”.

Preferably step (ii) involves noting the presence of an observable change that occurs due to the indicator compound when the threshold concentration of leukocytes is present. Preferably the observable change is a colour change.

In some embodiments step (ii) may be carried out immediately after step (i). However in preferred embodiments step (ii) is carried out after a period of incubation. During the incubation period the activity of the leukocyte cells causes a visible change in the indicator, preferably a colour change of a redox indicator.

The present invention relates to a method of determining the presence of a threshold concentration of leukocyte cells in a sample, suitably a sample of fluid from the peritoneal cavity preferably a sample of PD effluent.

The presence of leukocyte cells in a sample is often due to an infection, for example a microbial infection. In some embodiments the method of the present invention may further involve determining the presence of microorganisms in the sample.

In some embodiments of the first aspect of the present invention there is provided a method of analysing a sample to determine the presence of a threshold concentration of leukocyte cells and for the presence of microorganisms, said method comprising the steps of:
(i) contacting the sample with
   (I) a leukocyte detection means comprising:
      (a1) an indicator compound; and optionally
      (b) a buffer; and
   (II) a first reporting means comprising:
      (a2) an indicator compound; and
      (d1) media and/or nutrients that support or encourage microbial growth; and
(ii) examining the leukocyte detection means and the reporting means.

Preferably the present invention relates to a method of detecting leukocytes and microorganisms in a sample taken from a dialysis patient. Preferably the sample comprises peritoneal dialysis effluent.

In some embodiments the present invention involves analysis of a sample for the presence of microorganisms.

Throughout this specification reference is made to “microorganisms” and this term should be understood as encompassing all life forms not visible to the naked eye. As such, the term “microorganism” may include, for example, bacteria, fungi, viruses, protozoa and algae. It is preferred that the present invention may be used to identify detect and/or quantify one or more microorganisms selected from the group consisting of, bacteria, fungi, protozoa and algae. It is preferred that the present invention is used to detect bacteria and in particular pathogenic bacteria.

The invention may be used to detect the presence of Gram positive bacteria and/or Gram negative bacteria. Bacteria are classified as Gram positive and Gram negative organisms on the basis of staining characteristics.

By “Gram positive bacteria” we mean bacteria that have a thick peptidoglycan cell wall and no outer membrane, which therefore stain with crystal violet. In peritoneal dialysis, infection caused by Gram positive bacteria often indicates contamination of the dialysis catheter by skin commensals.

By “Gram negative bacteria” we mean bacteria that have an inner and outer membrane, and a thin peptidoglycan layer. These bacteria are therefore not able to retain the crystal violet stain.

It is most preferred that the present invention used to establish whether or not a peritoneal dialysis effluent is contaminated with one or more microorganisms selected from Staphylococcus aureus (and particularly multiresistant Staphylococcus aureus—MRSA), Pseudomonas aeruginosa, Staphylococcus epidermidis, Streptococcus mitis, Streptococcus sanguis, Enterococcus faecium, Escherichia coli, Enterobacter cloacae, Enterobacter aerogenes, Enterococcus faecalis, Klebsiella pneumoniae, Candida albicans, Acinetobacter baumannii, Stenotrophomonas maltophilia, Serratia marcescens, Proteus mirabilis, Bacillus cereus or Gram negative bacilli.

The present invention may involve contacting the sample with a first reporting means. By “reporting means” we mean to refer to a composition or components thereof which function to report the presence (or absence) of microorganisms in a sample.

Reference to “activation” or “triggering” of the reporting means refers to a change in the reporting means, suitably a positive result indicating the presence of microorganisms.

The first reporting means comprises (a2) an indicator compound.

The indicator compound may be any compound that undergoes an observable change when microorganisms are present.

The observable change may be a change in light absorption, precipitate formation, bubble formation, temperature change or other measurable quality.

Preferably the indicator compound (a2) is a colour change indicator. Preferably it is a redox indicator.

Examples of suitable indicators include Crystal violet, Carbol fuchsine, Safronin, Nigrosin, Indian ink, Iodine, Ziehl-Neelsen, Haemotoxylin, Eosin Y/Eosin yellowish, Papanicolaou, Orange G, Light green SF yellowish, Bismarck brown Y, Nile blue/Nile blue A, Nile red/Nile blue oxazone, Mason's trichome, Romanowsky, Wright's, Jenner's, Leishman, Giemsa, Silver, Sudan III, Sudan IV, Oil red O, Sudan Black B, Conklin, Malachite green, Osmium tetroxide/Tetraoxide, Rhodamine, Acridine Orange, Carmine, Coomassie blue, DAPI, Eosin B, Ethidium bromide, Acid fuchsine, Hoechst, Methylene green, Methylene blue, Neutral red/Toluylene red, and HDTMA/CTAB.

Examples of further indicators that may be used include Resazurim (e.g. Alamar blue) and 10-acetyl-3,7-dihydroxyphenoxazine (Amplex Red). It is preferred that the indicator is activated by an enzyme endogenous to the micro-organism being detected and more preferred that the indicator is activated by the action of a cellular reductase (e.g. an NAD(P)H reductase).

Preferably the indicator is a redox indicator.

Preferably the indicator compound is a tetrazolium compound.

Suitable tetrazolium compounds include MTT (3-(4,5-Dimethylthiazol-2-yl)-2,5-diphenyltetrazolium bromide); XTT (2,3-bis-(2-methoxy-4-nitro-5-sulfophenyI)-2H-tetrazolium-5-carboxanilide); MTS (3-(4,5-dimethylthiazol-2-yl)-5-(3-carboxymethoxyphenyl)-2-(4-sulfophenyl)-2H-tetrazolium); water soluble tetrazolium salts (VVST) such as WST-1, WST-3, WST-4, WST-5, WST-7, WST-8, WST-9, WST-10 or WST-11; indonitrotetrazolium chloride (INT); Nitrobluetetrazolium (NBT); Tetranitro blue tetrazolium (TNBT); Thiocarbamyl nitro blue tetrazolium (TCNBT); Tetrazolium red (TR); tetrazolium violet (TV) and neotetrazolium chloride; and 5-cyano-2,3-ditolyltetrazolium chloride (CTC).

More preferably the indicator compound (a2) is a water soluble tetrazolium salt.

Suitably the water soluble tetrazolium salt (WST) is selected from WST-1, WST-3, WST-4, WST-5, WST-7, WST-8, WST-9, WST-10 or WST-11.

It is most preferred that the indicator compound (a2) is WST-9 or a derivative thereof. WST-9 has the chemical formula: 2-(4-Nitrophenyl)-5-phenyl-3-[4-(4-sulfophenylazo)-2-sulfophenyl]-2H-tetrazolium, monosodium salt and the chemical structure:

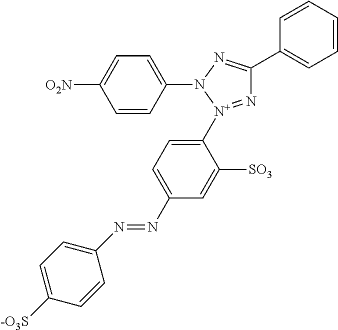

The first reporting means further comprises (d1) media and/or nutrients that support or encourage microbial growth.

The media and/or nutrients are included in the reporting means to encourage microorganisms to grow or multiply such that the reporting means may be triggered, typically by a change in colour of the indicator compounds.

During development work the inventors found that a number of factors can potentially lead to the degradation of the indicator compound or can lead to false triggering (i.e. the generation of the reporting signal in the absence of micro-organisms) of the indicator compound. The inventors found that selection of an appropriate media and/or nutrients that support or encourage microbial growth was a significant technical hurdle during the development of devices according to the invention.

Preferably the media and/or nutrients are selected to:
(i) maintain viable micro-organisms in the reporting means and in some embodiments supports or encourages microbial growth and/or division;
(ii) take into account whether a narrow spectrum or broad spectrum of microorganisms needs to be maintained and detected;
(iii) not result in false triggering of the indicator in the absence of a threshold concentration of microorganisms;
(iv) not degrade or inactivate any of the components of the reporting means.

A media and/or nutrients used in the reporting means is preferably a media or broth that does not cause the conversion of a WST into formazan when the media or broth is incubated with a WST at 37° C. overnight. Alternatively, the media and/or nutrients used in the reporting means is preferably a media or broth that does not cause the conversion of a WST into formazan when the media or broth is incubated with a WST at 24° C. for 8 to 24 hours. Conversion of a WST into formazan may be assayed by measuring, over time, the Optical Density of media solutions mixed with the WSTs.

Examples of media/nutrients which may be used according to the invention include Mueller Hinton, brain heart infusion broth (BHI) and Wilkins Chalgren media. Preferably the media/nutrients is selected from brain heart infusion and Wilkins Chalgren media. It is most preferred that Wilkins Chalgren media is used in the first reporting means.

In some embodiments step (i) of the method of the present invention may further comprise contacting the sample with (III) a second reporting means wherein the second reporting means comprises:

(a3) an indicator compound;

(d2) media and/or nutrients that support or encourage microbial growth; and

(e) a selection factor.

The indicator compound (a3) present in the second reporting means is suitably selected from the indicator compounds defined in relation to the first reporting means. The indicator compound used in the second reporting means may be the same as that used in the first reporting means or it may be different. Preferably the same indicator compound is used in the first reporting means and the second reporting means.

Preferably the indicator compound (a3) used in the second reporting means is a redox indicator. Preferably the indicator compound (a3) is a water soluble tetrazolium salt. Preferably it is selected from WST-1, WST-3, WST-4, WST-5, WST-7, WST-8, WST-9, WST-10 or WST-11. Most preferably the indicator compound (a3) present in the second reporting means is WST9.

The media and/or nutrients that support or encourage microbial growth in the second reporting means (d2) may suitably be selected from the media and/or nutrients defined in relation to the first reporting means.

The media and/or nutrients (d2) used in the second reporting means may be the same or different to those used in the first reporting means.

Preferably the same media and/or nutrients are used in the first reporting means and the second reporting means.

The media and/or nutrients that support or encourage microbial growth in the second reporting means is selected from brain heart infusion broth and Wilkins Chalgren media.

The second reporting means further comprises (e) a selection factor.

By “selection factor” we mean an agent that may be incorporated within the second reporting means that will arrest replication, decrease growth or increase death of certain microorganisms and will not affect the growth or death rate of others. It will be appreciated that a sufficient amount of the selection factor should be included in the reporting means that will prevent any activation of the device by microorganisms that are sensitive to it.

For the avoidance of doubt when we refer to activation of the first and/or second reporting means we mean that the indicator compound has undergone an observable change, suitably a colour change. The skilled person will appreciate that the observable change will occur once a threshold concentration of microorganisms is reached.

The skilled person will appreciate that the threshold concentration will depend on the constituents of the reporting means and the amounts thereof. These may be adjusted as appropriate by the skilled person.

The selection factors according to the invention are suitable for allowing discrimination between different types of microorganism when the activation of the first and second reporting means are compared.

In one embodiment a selection factor is chosen that has broad spectrum activity against bacteria, but which is selective for bacteria over other types of microorganism.

In another embodiment the selection factor may be an agent with narrow spectrum activity (for instance an agent that only has antibiotic activity against a limited number of species of bacteria). Such narrow spectrum selection factors are useful as selection factors when a device is designed where the user expects a sample to contain a specific microorganism. By way of example Sodium Nalidixate is a narrow spectrum agent which is used against Pseudomonas sp. It may be used as a selection factor in the second channel in devices designed to identify whether or not there is a Pseudomonas sp infection (activation of the first reporting means, but not the second reporting means will indicate this).

In a further embodiment the selection factor prevents the growth and/or metabolism of Gram −ve microorganisms. According to this embodiment, activation of both the first and second reporting means will indicate that a subject is infected with a Gram +ve microorganism because the selection factor in the second channel failed to prevent activation of the indicator compound. A user will establish that there is a Gram −ve infection if only the first reporting means (and not the second) is activated. Polymyxin B sulphate, gentamycin or monobactam compounds are antibiotics used primarily for Gram-negative infections and which may be used according to this embodiment of the invention.

Gram +ve microorganisms are a common problem in the development of peritonitis. Therefore, according to a preferred embodiment of the invention, the selection factor prevents the growth and/or metabolism of Gram +ve microorganisms. According to this embodiment, activation of both the first and second reporting means will indicate that a subject is infected with a Gram −ve microorganism because the selection factor in the second reporting means failed to prevent an absorbable change in the indicator compound. However, a user will establish that there is a Gram +ve infection if only the first reporting means (and not the second) is activated.

Fusidin (Fusidic acid) may be used as a selection factor for establishing whether or not there is a Gram +ve infection. It is a bacteriostatic antibiotic which is effective primarily against Gram-positive bacteria.

A preferred selection factor which inhibits the growth of Gram +ve organisms is vancomycin.

Other suitable antibiotics that may be useful include other glycopeptides, for example telavancin and teichoplanin; or lipopeptides, for example daptomycin.

In step (i) of the method of the present invention a sample is contacted with (I) a leukocyte detection means, optionally (II) a first reporting means, and optionally (III) a second reporting means.

The amount of indicator compound, media and/or nutrients and selection factor (as appropriate) in the first and second reporting means is selected to provide an appropriate final concentration in the resultant composition obtained after mixing the reporting means with the sample. These mixtures obtained upon admixture of the sample with a reporting means may be referred to herein as “the tested fluid”.

The amount of an indicator compound in the first and second reporting means will depend upon the size of the container in which it is provided and the volume of sample it is designed to retain for testing. Preferably a sufficient amount of indicator compound is included in the reporting means such that the final concentration of indicator compound (preferably WST-9) in the fluid being tested is greater than 0.01 mM and more preferably greater than 0.075 mM. Preferably the indicator compound is provided in an amount to provide a final concentration in the resultant composition of 0.075-1.5 mM, and more preferably in the range 0.1-12.0 mM.

For instance, WST-9 may be used in an amount to provide a final concentration in the range of 0.075-1.5 mM, and more preferably in the range 0.1-12.0 mM. Most preferred concentrations of the indicator compound (preferably WST-9) in the reporting means are in the range 0.2 mM-6.0 mM and particularly about 0.6 mM (e.g. 0.6±1.0 mM). At the most preferred concentration (0.6 mM), this equates to the inclusion of 0.38mg/mL of WST-9 in the tested fluid. In a preferred embodiment the reporting means is provided in a channel designed to receive about 16mL of fluid. Such channels therefore contain about 6.04mg of WST-9.

Preferably the media and/or nutrients that support or encourage microbial growth are present in an amount to provide a final concentration in the range 1-50g/L in the fluid being tested, preferably in the range 2-40g/L. In one embodiment about 33g/L Wilkins Chalgren media may be found in the fluid (the recommended concentration of the media). However, in preferred embodiments between about 2-18g/L may be used as a final concentration in the fluid (e.g. 6.6g/L). By way of example, a channel designed to receive 16mL of fluid will ideally contain about 100-500mg of Wilkins Chalgren media.

The inventors have found that a concentration of between 1 ng/mL and 1mg/mL of fluid is a suitable final concentration of selection factor in a test fluid, for example from 0.1 to 100 μg/mL fluid. The amount of selection factor (preferably an antibiotic) needed will depend on the nature of the compound and its potency. In one preferred embodiment in which the selection factor is vancomycin the concentration is preferably between 5 and 40 μg/mL, for example about 16 μg/mL. By way of example, a channel designed to receive 16mL of fluid will ideally contain about 256 pg of vancomycin.

The first and second reporting means compositions may optionally comprise one or more further components.

In some preferred embodiments the first reporting means comprises an electron mediator. In some preferred embodiments the second reporting means comprises an electron mediator.

Suitable electron mediators are as defined in relation to the leukocyte detection means.

Preferred electron mediators include menadione and phenazine electron mediators.

Preferably the electron mediator (c2) of the first reporting means is menadione or mPMS.

Preferably the electron mediator (c3) and the second reporting means is menadione or mPMS.

The electron mediator (preferably mPMS) is preferably included in the first and/or second reporting means such that its final concentrations are greater than 0.001 mM in the tested fluid. For instance, the electron mediator (preferably mPMS) may be used in the first and/or second reporting means in the range of 0.001-0.1 mM, and more preferably in the range 0.005-0.05mM. In one embodiment a channel designed to receive 16 mL of fluid will ideally contain about 100 to 300 μg of mPMS.

In step (i) of the method of the present invention, the sample is contacted with:
(I) a leukocyte detection means comprising
(a1) an indicator compound; and optionally
(b) a buffer; and optionally
(II) a first reporting means comprising:
(a2) an indicator compound; and
(d1) media and/or nutrients that support or encourage microbial growth;
and optionally
(III) a second reporting means comprising:
(a3) an indicator compound;
(d2) media and/or nutrients that support or encourage microbial growth;
and
(e) a selection factor.

In some embodiments the first step (i) of the first aspect of the invention involves contacting the sample with the leukocyte detection means (I) and the first reporting means (II).

In some embodiments the first step (i) of the first aspect of the invention involves contacting the sample with the leukocyte detection means (I) and the second reporting means (III).

In preferred embodiments step (i) involves contacting the sample with the leukocyte detection means (I), the first reporting means (II) and the second reporting means (III).

In step (i) of the method of the present invention, the sample is preferably contacted with:
(I) a leukocyte detection means comprising
(a1) an indicator compound; and optionally
(b) a buffer.
(II)a first reporting means comprising:
(a2) an indicator compound;
(d1) media and/or nutrients that support or encourage microbial growth;
and
(c2) an electron mediator;
(III) a second reporting means comprising:
(a3) an indicator compound;
(d2) media and/or nutrients that support or encourage microbial growth;
(e) a selection factor; and
(c3) an electron mediator.

In some preferred embodiments the present invention provides a method of analysing a sample, preferably a sample taken from a dialysis patient, for the presence of threshold concentration of leukocytes cells and for microorganisms, said method comprising the steps of:
(i) contacting the sample with
   (I) a leukocyte detection means comprising:
   (a1) an indicator compound; and
   (b) a buffer;
(II) a first reporting means comprising:
   (a2) an indicator compound;
      (d1) media and/or nutrients that support or encourage microbial growth; and
   (c2) an electron mediator; and
   (III) a second reporting means comprising:
   (a3) an indicator compound;
      (d2) media and/or nutrients that support or encourage microbial growth;
   (e) a selection factor; and
(c3) an electron mediator; and
(ii) examining the leukocyte detection means and the reporting means.

The method of the first aspect of the present invention further includes the step (ii) of examining the leukocyte detection means and when present, the reporting means. This is suitably carried out to determine whether leukocyte cells or microorganisms are present.

The skilled person will appreciate that this examination will be of the resultant composition that is obtained when the leukocyte detection means and reporting means have been contacted with the sample. This may also be referred to herein as “the tested fluid”.

In some embodiments step (ii) involves examining the first reporting means and the leukocyte detection means.

Most preferably step (ii) involves examining the leukocyte detection means, the first reporting means and the second reporting means.

The leukocyte detection means (I) and when present the first and/or second reporting means (II) and (III) should be exposed to the sample fora period of time and under conditions suitable to facilitate, encourage or cause any microorganisms and leukocytes present in the sample to pass into the reporting means and leukocyte detection means. Microorganisms that have successfully passed into the reporting means may be maintained or multiply in the media and/or nutrients that support or encourage microbial growth. At this point, means (I), (II) and (III) may be left in contact with the sample to allow the reporting means to complete any reactions necessary to report the presence of microorganisms to the user. Alternatively, after a period of incubation with the sample, the sample could be separated from the leukocyte detection means and, when present, the first and second reporting means and incubated for a further period of time before the reporting means and leukocyte detection means are examined.

It will be appreciated that the length of incubation will depend the nature of the PD fluid being tested and also the temperature at which incubation occurs. Preferably the compositions should be incubated for at least 2 hours and preferably at least 4 hours. Such compositions may be typically incubated for 4-24 hours, preferably 4 -18 hours and more preferably 4-12 hours. Preferred incubation times are 4 hours, 6 hours, 8 hours, 10 hours or 12 hours.

The composition(s) may be incubated at ambient room temperature. In one embodiment of the invention the composition(s) may be incubated at 20° C., 30° C. or more preferably 37° C. In a preferred embodiment the composition(s) may be placed in, or on, an incubator which will maintain the composition(s) at a desired incubation temperature (e.g. 37° C.).

Incubation is suitably carried out at a temperature of 30 to 39° C., preferably about 37° C., preferably for a period of 4 to 10 hours, preferably 8 to 12 hours. In some embodiments the device may be shaped to fit within an incubator.

Preferably the compositions are cooled following incubation, for example to about 4° C. This cooling inhibits microbial activity and thus reduces further activation of the compositions after incubation.

Preferably step (ii) involves examining the colour of the leukocyte detection means and when present the colour of the first reporting means and/or second reporting means.

In some embodiments the colour of the leukocyte detection means and, when present, the first and/or second reporting means may be compared with a colour chart. When the first and/or second reporting means and/or leukocyte detection means are provided within a device or devices, the colour chart may form part of the device or devices.

In preferred embodiments a selection factor in the second reporting means prevents the growth of Gram +ve microorganisms and allows the user of the device to discriminate between Gram +ve and Gram −ve infections. Activation of neither the first or second reporting means informs a user that there is no microbial contamination of the sample (eg PD effluent) or that the titre of microorganism in the sample (eg PD effluent) is below clinically significant levels. Such levels will depend on the particular microorganisms but for those microorganisms commonly found in PD effluent, clinically significant levels are typically 10⁴ cfu/mL, 10⁵ cfu/mL or above.

Activation of the first reporting means informs a user that the sample (eg PD effluent) is contaminated with a microorganism. Activation of the first reporting means, and not the second reporting means, indicates that the microbial contamination is Gram +ve whereas activation of both the first and second reporting means, indicates that the microbial contamination includes microorganisms other than Gram +ve bacterium (whether or not Gran +ve microorganisms are also present).

Activation of the leukocyte detection means indicates that leukocytes are present in a greater concentration than the threshold concentration, which is indicative of infection.

In some embodiments the leukocyte detection means may comprise an antibacterial agent which prevents the growth, metabolism and/or multiplication of bacteria. Any agent which selectively inhibits bacteria in the presence of leukocytes can be used. This may help ensure that any bacteria present in the leukocyte detection means do not trigger these means.

Suitable antibacterial agents will be known to the person skilled in the art and include, for example, broad spectrum antibiotics.

Examples of suitable antibacterial agents include penicillins and penicillin combinations, meropenem, chloramphenicol, second and third generation cephalosporins, erythromycin, first generation cephalosporins (i.e cephalexin daptomycin glycopeptides for example vancomycin), ciprofloxacin and aminoglycosides, for example gentamycin.

In one embodiment the leukocyte detection means comprises the antibacterial agents meropenem, ciprofloxacin and vancomycin.

These will suitably be included in an amount to provide from 0.01 to 1 mg/mL.

In some embodiments the first reporting means and/or second reporting means may further comprise a leukocyte inhibiting agent which prevents the growth and/or multiplication of leukocytes. Any agent which selectively combats leukocyte cells in the presence of bacteria may be used. This may help ensure that any leukocyte cells present in the first or second reporting means do not trigger these means. Suitable leukocyte inhibiting agents include saponins and surfactants.

Suitable saponins include digitonin.

Suitable surfactants include anionic, non-ionic and amphoteric surfactants.

Suitable anionic surfactants include salts of alkyl sulfates, alkyl ether sulfates, fatty acids, carboxylates, alkyl or aryl sulfonates, isethionates, alkyl phosphates, sulfosuccinates, taurates, sarcosinates, sulfoacetates, lactates, acyl amino acids and phosphonates.

Suitable non-ionic surfactants include fatty alcohols, alkoxylated alcohols, alkoxylated phenols, alkyl amine oxides, alkyl phosphine oxides, alkyl sulfoxides, sorbitan and sucrose esters, alkylpolyglucosides and alkoxylated alkylpolyglucosides.

Suitable amphoteric surfactants include alkyl betaines, alkyl sultaines and amphoacetates.

Preferred surfactants are anionic surfactants, especially sulfate compounds.

One preferred leukocyte inhibiting agent is sodium dodecyl sulphate (or SDS).

The anionic surfactant may suitably be provided in an amount of from 0.001 to 5 wt% based on the amount of tested fluid, preferably from 0.01 to 1 wt%.

In some preferred embodiments the present invention provides a method of analysing a sample, preferably a PD effluent sample taken from a dialysis patient, for the presence of threshold concentration of leukocytes cells and for microorganisms, said method comprising the steps of:
(i) contacting the sample with
   (I) a leukocyte detection means comprising:
      (a1) an indicator compound;
      (b) a buffer;
      (c1) an electron mediator; and
      (f) an antibacterial agent;
   (II) a first reporting means comprising:
      (a2) (a2) an indicator compound;
      (d1) media and/or nutrients that support or encourage microbial growth;
      (c2) an electron mediator;
      (g1) a leukocyte inhibiting agent; and
   (III) a second reporting means comprising:
      (a3) an indicator compound;
      (d2) media and/or nutrients that support or encourage microbial growth;
      (e) a selection factor;
      (c3) an electron mediator; and
      (g2) a leukocyte inhibiting agent; and
   (ii) examining the leukocyte detection means and the reporting means.

Compositions (I) and (II) and/or (III) when used in the method may be provided separately in individual containers.

Composition (I) and (II) and/or (III) when present may be provided separately or may be provided as part of the same device.

According to a second aspect of the present invention there is provided a device for detecting a threshold concentration of leukocytes in a sample, the device comprising a channel arranged to receive the sample wherein the channel contains a leukocyte detection means comprising (a1) an indicator compound.

Suitably the method of the first aspect may be carried out using a device of the second aspect.

Preferred features of the second aspect are as defined in relation to the first aspect. Further preferred features of the first and second aspects will now be described.

In some embodiments the device of the second aspect comprises a further channel which comprises a reporting means that can detect and/or identify microorganisms in the sample, said reporting means comprising (a2) an indicator compound; and (d1) media and/or nutrients that support or encourage microbial growth.

It will be appreciated that the device according to the invention may be used to detect or identify leukocytes and microorganisms in a variety of different fluids. It is preferred that the device is used to test a biological fluid (e.g. bronchial lavage fluid, serum, cerebral spinal fluid, urine and the like) and it is most preferred that the device is used to detect or identify microorganisms in PD effluent.

In some embodiments the device of the second aspect may be useful for determining the presence of a threshold concentration of leukocytes in a sample and for detecting and/or identifying microorganisms in the sample, said device comprising three channels that are arranged to receive the sample and wherein:

a first channel contains a first reporting means comprising:
(a2) an indicator compound; and
(d1) media and/or nutrients that support or encourage microbial growth; and

a second channel contains a second reporting means comprising:
(a3) an indicator compound;
(d2) media and/or nutrients that support or encourage microbial growth; and
(e) a selection factor which selectively inhibits growth of microorganisms; and

a third channel contains a leukocyte detection means comprising:
(a1) an indicator compound; and
(b) a buffer.

Preferably the first reporting means present in the first channel is as defined in relation to the first aspect. Preferably the indicator compound is a water soluble tetrazolium salt.

Preferably the first reporting means further comprises (c2) an electron mediator.

Preferably the second reporting means composition present in the second channel is as defined in relation to the first aspect. Preferably the indicator compound is a water soluble tetrazolium salt.

Preferably the second reporting means further comprises (c3) an electron mediator.

Preferably the leukocyte detection means provided in the third channel is as defined in relation to the first aspect.

Preferably the leukocyte detection means further comprises (b) a buffer.

In some embodiments the leukocyte detection means may further comprise (c1) an electron mediator.

In some embodiments the first reporting means may further comprise a leukocyte inhibiting agent.

In some embodiments second reporting means means may further comprise a leukocyte inhibiting agent.

In some embodiments the leukocyte detection means may further comprise an antibacterial agent.

Other preferred features of the second aspect are as defined in relation to the first aspect.

Further preferred features of the first and second aspects will now be defined.

Preferably, the first aspect of the present invention provides a method of analysing a sample taken from a dialysis patient for the presence of a threshold concentration of leukocytes and in preferred embodiments microorganisms, said method comprising the steps of:
(a) contacting a device provided by the second aspect of the invention with the sample to be analysed; and
(b) examining the reporting means.

The channels of the device may be any suitable vessel that can retain components of the reporting means and into which a sample fluid may be introduced. The device should also be designed such that the contents of channels may be easily observed by a user of the device.

In a preferred embodiment each channel is a bag which contains the reporting means and wherein each bag has a tube connected to it for receiving the fluid. Such bags may be formed from a number of materials that are well known to the art and in a preferred embodiment such bags are formed from PVC.

It is preferred that the bags are formed by sealing two sheets of PVC together with components of the reporting means placed between the two sheets before sealing. At least one of the sheets should be transparent (for viewing the contents) and it is preferred that one sheet is transparent and the other sheet opaque and preferably white.

FIG. 2 illustrates one embodiment of the invention in which the channels comprise bags and the examples describes how such bags may be formed.

It will be appreciated that the channels, particularly when they are bags connected to tubing (as illustrated in FIG. 2) are ideally contained within a suitable casing.

In some preferred embodiments the casing incorporates a colour chart that enables a user to compare the colour of the leukocyte detection means and, when present, first and/or second reporting means with the colour provided on the chart. The colours on the chart will illustrate the colour to be expected in the presence/absence of leukocytes and/or microorganisms.

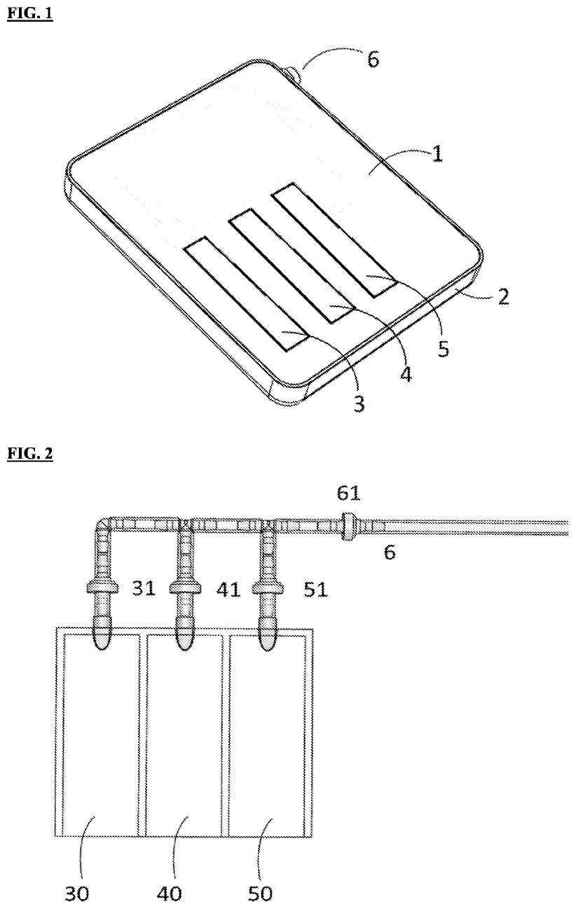

FIG. 1 illustrates the sort of casing/container which may be used to retain the channels. The container has three viewing windows on the top surface which are aligned over the reporting means and leukocyte detection means in the channels to allow a user to observer whether or not the fluid in the channels is infected by a micro-organism. In a preferred embodiment components of the reporting means and leukocyte detection means are contained within capsules. These capsules dissolve to release their contents into the fluid when the fluid is introduced into the channels. When the indicator compound is a WST compound a user of the device will observe a clear or straw-coloured fluid if the fluid is not infected/contaminated whereas the fluid in the first and/or second channels will turn a dark/purple colour (the WST is reduced to formazan) if microorganisms infect the fluid. Where MTT is the indicator compound used, the leukocyte channel will turn blue if leukocyte cells indicating infection are present.

The assembly of a device with a most preferred casing which is in the form of a thermoformed blister tray with a Tyvek lid is later described in the examples.

Fluids may be applied to each of the channels of the device in a number of ways. For instance, in embodiments of the invention where the channels are bags with tubes attached, a sterile syringe may be used to draw up a fluid sample and the fluid inserted into the channel by attaching the syringe to the tube. The fluid in the channels will then allow the components of the reporting means to mix and any microorganisms in the fluid will cause the indicator to undergo, for example a colour change, after suitable period of incubation. Alternatively fluid may be pumped into the device or even enter by gravity (i.e. the fluid drains into a device placed lower than the fluid container).

Components of the reporting means and leukocyte detection means (for example the indicator compounds, media and/or nutrients that support or encourage microbial growth etc.) may comprise powders that are inserted directly within the channels. For instance, the channels may comprise bags into which each component of the reporting means or leukocyte detection means is injected.

However, it is preferred that each channel/bag contains the leukocyte detection or reporting means, or individual components thereof that are loaded onto, or into, some kind of vehicle. Such vehicles are useful for designing an optimal method of manufacturing devices according to the invention and can be particularly useful when the device, or at least components of the reporting and/or leukocyte detection means, need to be sterilized.

A number of vehicles may be used. For instance, components of the reporting or leukocyte detection means may be made into concentrated solutions that are applied to filter discs. The filter discs are then dried such that they retain the relevant component and the filter discs then placed within the channels. According to one embodiment the selection factor (e.g. vancomycin) may be applied to a filter disc. By way of example, 6 mm Whatnnan or Oxoid filter discs may be impregnated with 20p1 of a concentrated stock of the reporting means or leukocyte detection means component (e.g. indicator compound, a selection factor or electron mediator). These discs should then be dried (e.g. at 37° C. for 18 hours or until completely dry). The dried discs may then be inserted in the relevant channels. Alternatively the discs may be soaked in a concentrated stock solution. In some embodiments a commercially available impregnated filter disc could be used.

Alternatively, components of the reporting means or leukocyte detection means may be combined with suitable binders and excipients to form tablets and the tablets placed within the channels.

It is preferred that the vehicle for the reporting means, the leukocyte detection means or components thereof, is a capsule or capsules. In one embodiment all components of one channel are retained within one capsule. In another embodiment a channel may contain more than one capsule with components of the reporting means or leukocyte detection means contained within different capsules.

Capsules used according to the invention should dissolve when contacted by the fluid being tested and are also ideally colourless or at least a colour that does not affect the visualisation of the reporting means. Capsules are well known to the art and a skilled person will be easily able to select a capsule which suits the particular channels into which they will need to be inserted. Preferred capsules may be formed from hydroxypropyl methylcellulose (HPMC) or gelatine.

The size of capsule used will depend on the amount of reporting means or leukocyte detection means (or components thereof that need to be introduced into the channel); and this in turn will depend upon the size of the channel and the amount of fluid it is designed to retain. By way of example the inventors have found that size 5 Capsugel Vcap capsules may be suitably used in channels designed to receive around 16 mL of test fluid.

In one embodiment all components of the first or second reporting means are retained within a single capsule. For instance, a capsule may contain WST-9, Wilkins Chalgren media, mPMS and optionally vancomycin.

In one embodiment the leukocyte detection means is retained within a capsule for use in a channel designed to receive around 16 mL test fluid.

In some embodiments the components of the first and/or second reporting means and/or leukocyte detection means are mixed with excipients and then used to fill capsules. A preferred excipient is polyvinylpyrrolidone (PVP) or a derivative thereof. PVP is preferred as it did not cause false triggering or mask a colour change. In fact, to their surprise, the inventors found that PVP seemed to improve and intensify the colour change which occurs when WST-9 is reduced to formazan.

In some preferred embodiments polyvinylpyrrolidone (PVP) is used as an excipient. This may be for example under circumstances where components of the reporting means need to be mixed with an excipient (e.g. when tablets or formed or to aid in the filling of capsules).

In some embodiments the first reporting means comprises polyvinylpyrrolidone.

In some embodiments the second reporting means comprises polyvinylpyrrolidone.

In some embodiments the leukocyte detection means comprises polyvinylpyrrolidone.

The final concentration of PVP in the tested fluid is preferably greater than 0.25% (w/v) PVP and more preferably should be at least 0.8% (w/v) PVP. According to one embodiment of the invention the final concentration of PVP should be about 1.25% (w/v). In another embodiment of the invention the final concentration of PVP may be up to about 3.0% (w/v). A skilled person will appreciate that the amount of PVP used as an excipient in capsules and the like may be adjusted with a view to the final concentration of PVP being in these preferred ranges.

In some preferred embodiments the first channel comprises a first reporting means comprising Wilkin Chalgren media, WST-9, mPMS and PVP; the second channel comprises a second reporting means comprising Wilkins Chalgren media, WST-9, mPMS, vancomycin and PVP; and the third channel comprises a leukocyte detection means comprising MTT and MES buffer.

In some preferred embodiments the first channel comprises a first reporting means comprising Wilkin Chalgren media, WST-9, mPMS, PVP and digitonin; the second channel comprises a second reporting means comprising Wilkins Chalgren media, WST-9, mPMS, vancomycin, PVP and digitonin; and the third channel comprises a leukocyte detection means comprising WST-9, mPMS, PVP, MES buffer, meropenem, ciprofloxacin and vancomycin.

The components of each channel may be provided in any suitable form. They may be provided as a powder, tablet, gel, solution or paste.

Preferably each component is provided in powdered form within a capsule. Each capsule may comprise one or more of the individual components.

The first channel may comprise one or more capsules.

The second channel may comprise one or more capsules.

The third channel may comprise one or more capsules.

In some preferred embodiments all of the components of the first reporting means are provided in a single capsule.

In some preferred embodiments all of the components of the second reporting means are provided in a single capsule.

In some preferred embodiments all of the components of the leukocyte detection means are provided in a single capsule.

In some embodiments each channel comprises a single capsule.

In one embodiment of the invention the first channel contains a first reporting means comprising:
(1) a capsule containing Wilkins Chalgren media;
(2) a capsule containing a mixture of WST-9 and PVP; and
(3) a capsule containing a mixture of mPMS and PVP; and

the second channel contains a second reporting means comprising:
(1) a capsule containing Wilkins Chalgren media;
(2) a capsule containing a mixture of WST-9 and PVP;
(3) a capsule containing a mixture of mPMS and PVP; and
(4) a filter paper impregnated with vancomycin; and

the third channel comprises a leukocyte detection means comprising:
(1) a capsule containing MTT; and
(2) a capsule containing MES buffer.

The device according to the second aspect of the invention may be used to simply detect infection at an early stage. This means that treatment can be started more quickly, and so the infection may be controlled more easily.

When the sample is a clinical sample, as is preferred, early detection of a microorganism allows earlier treatment of a subject and this in turn reduces the morbidity and mortality associated with an infection (e.g. peritonitis). Early treatment also benefits health services because early treatment reduces the need for hospitalisation and thereby saves expense. Furthermore, when PD effluent is tested, the control and/or prevention of peritonitis allows patients to be maintained on PD.

The device according to the second aspect of the invention also advantageously provide additional information about the type of microorganism that is in the fluid. This information is important when a clinician wishes to choose the best treatment (e.g. when the device is used to test a clinical sample such as PD effluent).

Preferred uses of the present invention are for testing clinical fluid samples and in particular in) clinics, at a patient's home and other places which are “point of care”.

A most preferred use of the present invention is for testing PD effluent to assess whether or not a patient is developing or has developed peritonitis. PD effluent may be collected and then tested on a ward or even sent off for testing in a laboratory. However, it is preferred that devices are integrated into the routine a patient follows when removing PD effluent which has been resident in their abdomen for the required amount of time.

It is preferred that devices according to the invention are adapted such that they may be used with or even integrated with the procedures followed for Continuous ambulatory peritoneal dialysis (CAPD). CAPD uses gravity to drain the fluid out of the peritoneal cavity and replace it with fresh fluid. Each exchange takes around 30 minutes and most patients need to do 4 exchanges per day.

It is most preferred that devices according to the invention are adapted such that they may be used with or even integrated with the equipment used in Automated peritoneal dialysis (APD). APD is usually conducted at night using a machine that moves fluid in and out of the abdomen whilst the patient is asleep, usually over an 8 to 9 hour period. The machine is small enough to sit on top of a bedside table. Devices according to the invention are preferably designed such that they may fit to the effluent line from such machines and can therefore test the effluent for microbial contamination before the effluent is pumped to waste.

In preferred embodiments the channels of the device of the present invention are provided within a casing. Suitably the casing has one or more viewing windows which allow observation of the contents of the channel.

In some preferred embodiments in which the device has three channels, three viewing windows are provided, one for each channel.

In some preferred embodiments the casing includes a colour chart which allows a user to compare the colour visible in each channel with the colour on the chart. This suitably provides an indication of whether microorganism and/or leukocytes are present in the channel.

Suitably the colour chart may be provided in an area adjacent to the viewing window so that the colour chart and the channel are side-by-side.

In preferred embodiments the invention is substantially as described in the description and figures.

### DETAILED DESCRIPTION

The present invention will now be described in detail with reference to the following figures which show:

FIG. 1: represents a perspective view from top and two sides of a device which may be used according to the invention.

FIG. 2: represents a perspective view of channels within the device of FIG. 1.

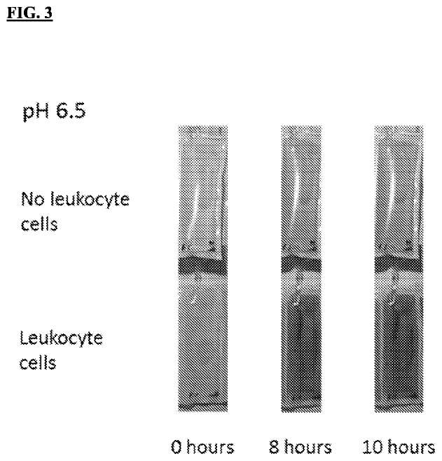

FIG. 3 shows a channel comprising a leukocyte detection means before incubation and after 10 hours of incubation.

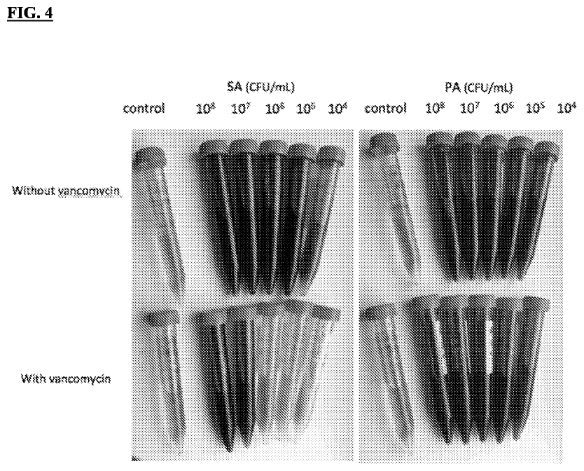

FIG. 4: is a photograph of tubes containing reporting means and no microorganisms (control) or between 10⁴ and 10⁶ CFU/mL of Staphylococcus aureus (SA) or Pseudomonas aeruginosa (PA). The upper row of tubes did not contain any selection factor whereas the lower row contained vancomycin.

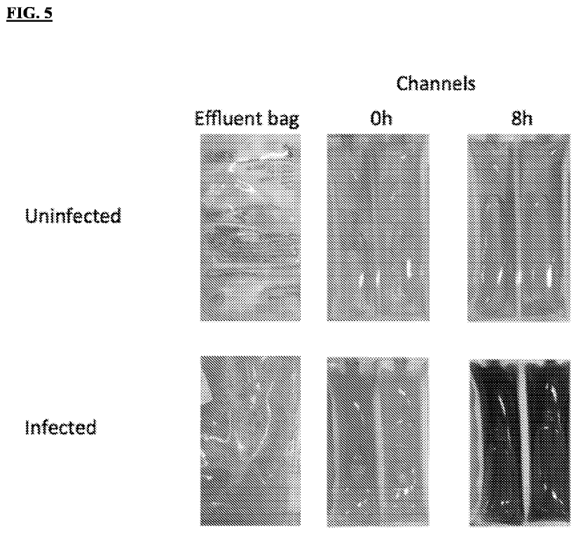

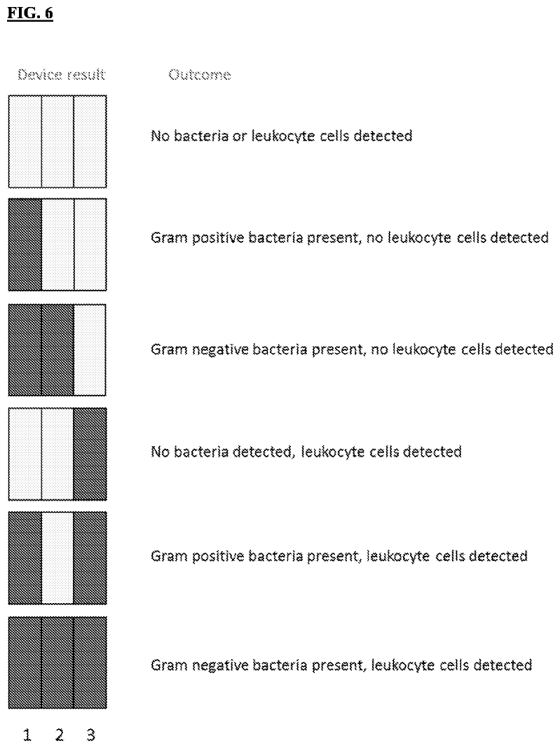

FIG. 5: the top row of photographs shows the appearance of an uninfected effluent bag and images of channels 1 and 2 (first and second reporting means) after 0 and 8hrs incubation with the PD effluent from the effluent bag; and the bottom row of photographs shows the appearance of an effluent bag which a clinician suspected was infected and images of channels (removed from the device casing) after 0 and 8hrs incubation with the PD effluent from the suspect effluent bag.

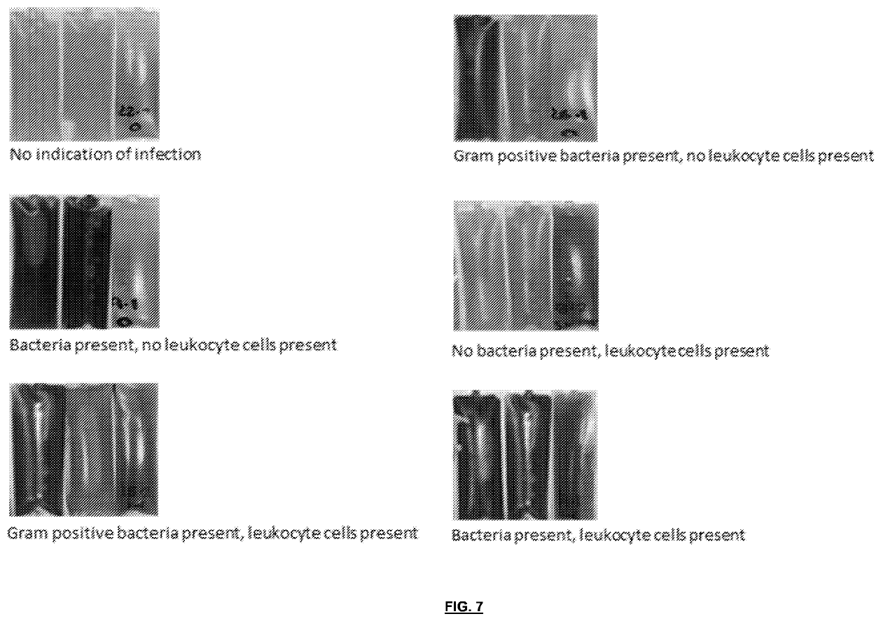

FIG. 6 is a schematic diagram showing possible outcomes for each channel and what this is indicative of, in terms of infection.

FIG. 1 is a perspective view from top and two sides of a device 1 according to the present invention. The device comprises an opaque plastic casing 2 which has transparent viewing windows 3, 4, 5 in the top face. A first viewing window 3 is aligned over a first channel 30 contained within the casing, the second viewing window 4 is aligned over a second channel 40 contained within the casing, and the third viewing window 5 is aligned over a third channel 50 contained within the casing. The viewing windows 3, 4, 5 are position such that the contents of the channels may be observed by a user of the device. In use fluid is introduced via inlet 6 and flows through tubing (not shown in FIG. 1) into the channels (also not shown in FIG. 1) which contain the reporting means.

FIG. 2 is a perspective view of the channels 30, 40, 50 contained within the casing 2 shown in FIG. 1. Tubing communicates fluid to the first channel 30, second channel 40 and third channel 50 which in this embodiment are bags with a transparent upper face. The tubing includes one-way valves, 31, 41, 51, 61 which prevent back flow of the fluid and reporting means up the tubing. The reporting means within the first channel 30 comprises one or more capsules containing WST9, mPMS Wilkins Chalgren media, and polyvinyl pyrrolidone (PVP) filler. The reporting means within the second channel 40 comprises one or more capsules containing WST9, mPMS, Wilkins Chalgren media, PVP filler and vancomycin is also provided impregnated on a filter disc. Each of the capsules dissolves when fluid is introduced into the channels. The components of the reporting means effectively combine when the fluid is introduced into the channels. The WST9 in the first channel is reduced to dark coloured formazan if microorganisms are present in the fluid and WST9 in the second channel is reduced to dark coloured formazan if microorganisms are present in the fluid which are resistant to vancomycin.

The leukocyte detection means 50 comprises in one or more capsules MTT and MES buffer.

### EXAMPLES

The inventors realised that there were no commercially available products that were small and simple which may be used for detecting high levels of leukocytes and microbial contamination of fluids, in particular, PD effluent.

Initial proof of principle experiments were conducted to establish whether or not devices could be created that could distinguish/select between microorganisms and also for which the threshold for triggering of the leukocyte detection means and reporting means could be controlled.

1. Preparation of Components of Reporting Means

Reporting means comprising WST-9, mPMS and PVP were tested in the presence of eight different species of bacteria. It was established that 10⁵ CFU/mL of all bacteria were able to reduce the tetrazolium to provide a colour change within 8 or 10 hours at 37° C.

Capsule Filling

An empty capsule was weighed as a blank. The tapped density of each active component (i.e. WST-9, mPMS and vancomycin) with PVP excipient was established by weighing the amount of active and adding it to a size 5 capsule. PVP excipient was added and compressed into the capsule until full. The capsule was weighed again to establish the mass of excipient required to fill a size 5 capsule for each active component. This was repeated 5× to give an average weight of a capsule for each active and excipient.

100× mass of each active and excipient was prepared and mixed thoroughly to generate a homogenous mixture of powders with the active evenly distributed throughout the excipient. This was used to fill 100 capsules using a Feton Fastlock Kit.

Preparation of a Vancomycin Filter Disc

6 mm Whatman filter discs were prepared by soaking in a stock of vancomycin in water. These were dried as a single sheet at 37° C. for 18 hours or until completely dry. The final concentration of antibiotic on each disc was 256pg.

2. Assembly of a Device according to the Invention

Device for detecting microbial contamination of PD effluent from an Automated peritoneal dialysis (APD) machine (Baxter) were produced by:

3. Fabrication of Channels and Loading them with Reporting Means
1. White PVC material is overlaid with transparent PVC, both cut to 93 mm×70 mm.
2. The longest edges are welded and two channels are created by welding the middle of the bag.
3. At the top edge two tubes (3.0 mm diameter, 25 mm length) are inserted, one into each channel, which are secured by sealing around the tubes.
4. Capsules are inserted between the PVC sheets that will form the first channel and the bottom edge is sealed.
5. Capsules and a vancomycin filter disk are inserted between the PVC sheets that will form the second channel and the bottom edge is sealed.
6. Capsules are inserted between
7. White PVC one way check valves are inserted into each of the tubes
8. Two flexible PVC tubes are secured to each valve. The two tube are joined by a PVC Y connector from which a single tube, 1 meter in length, is attached.

4. Assembly of the Device
9. The bag/channel assembly is inserted into a casing comprising a thermoformed blister tray and the assembly is secured in place by pressing the valves into preformed recesses in the blister tray.
10. The tube is fed through a hole in the right side of the blister tray.
11. At the end of the tube a valve in attached followed by a male luer lock that is compatible with the peritoneal dialysis consumables from which effluent is sampled.
12. The luer lock is protected by a cap.
13. The blister pack is sealed with an opaque Tyvek lid which has two transparent windows through which the bag channels can be visualised.
14. The blister tray and tubing is sealed into a PET/PE/Tyvek peel pouch and packaged into boxes.

5. Testing Channels for use in Devices according to the Present Invention

Dialysis effluent samples were obtained from peritoneal dialysis patients by filling their peritoneum with dialysate for a minimum of two hours. After this dwell time, fluid was drained from the peritoneum and a sample was tested by filling the channels of the device with 16mL per channel. The channels were then incubated at 37° C. for 8 hours, after which the result was read.

A leukocyte detection channel according to the invention was also prepared. This contained MTT buffered to pH 6.5 with MES. The PD effluent samples were tested using this channel. A first sample from a well-patient of clear fluid and independently confirmed to have below the threshold concentration of leukocyte cells was tested along with a cloudy sample of an unwell patient independently confirmed to contain high concentration of leukocytes. FIG. 3 shows in the top row the sample of the well patient initially and after incubation for 8 and 10 hours. In the bottom row the sample from the unwell patient is shown.

FIG. 5 shows photographs of first and second channels of two exemplary tests performed on PD effluent bags from a clinic where the patients were undergoing automated peritoneal dialysis (APD).

The top row of photographs shows the appearance of the channels from PD effluent collected from a patient that seemed well. Photographs were taken of the channels after 0 and 8hrs incubation at 37° C. and it can be seen that the reporting means within the channels have not been activated after 8 hours incubation. A photograph was also taken on the effluent bag and it can be seen that the fluid is relatively clear. These results suggested that the patient was not suffering from peritonitis and the clinician subsequently confirmed that the patient remained well.

The bottom row of photographs shows the appearance of the channels from PD effluent collected from a patient who had started to feel unwell. Photographs were also taken of the channels after 0 and 8hrs incubation at 37° C. A photograph was also taken on the effluent bag and it can be seen that the fluid did appear to be cloudy. The figure shows that the reporting means within both the first and second channels had activated. This suggested that the patient was suffering from peritonitis and that it was likely to be caused by a Gram −ve organism (Vancomycin in the second channel failed to inhibit reporter activation). Two days later the clinician confirmed that the patient had a Gram −ve infection when he received confirmation from the hospital testing laboratory. It will be appreciated that the device accurately and quickly (2 days quicker than routine laboratory testing) identified an infection and also the type of infection. This has the great advantage that clinicians may use the device to make informed and early decisions about treatment for peritonitis. This in turn improves the outcome for the patient, saves the health service money and also has the advantage that patient who can have infections identified and treated at an early stage have a better chance of being maintained on PD (rather than needing to be transferred to HD).

6. Use of Devices according to the Invention in a Clinical Setting.

A protocol was established, and followed, for when devices according to the invention were used to monitor for microbial contamination of PD effluent in conjunction with an Automated peritoneal dialysis (APD) machine (Baxter).

Users of the device were instructed to:
1. Wash and dry hands thoroughly and use aseptic technique to reduce the risk of contaminating the sampling device
2. Open the device packaging
3. Ensure the clamp on the effluent sample line is closed
4. Remove the cap from the sample line
5. Remove the red cap from the device. Save the cap.
6. Connect the device to the sample line with a twist action until locked
7. Position the device on the floor at the position of the drain bag
8. Follow the instructions on the Baxter machine when you start your treatment
9. At the point of ‘Initial drain’, wait for 40 seconds
10. Open the clamp on the sample line and allow the effluent to fill the device. This should take no more than 2 minutes.
11. When the device is full, close the clamp on the sample line.
12. Disconnect the device from the sample line and re-cap the connectors
13. Ensure the incubator is switched on at the plug
14. Take the sampling device to the incubator and place inside.
15. Close the door and press start
16. The device will heat to 37° C. and will stay incubated at this temperature for 10 hours, after which it will cool to 4° C. (fridge temperature).
17. You can view the device after a minimum of 10 hours.
18. If you are not ready to view the device immediately, keep the device in the incubator with the door closed which will keep the result fixed. If you remove the device it must be read within 1 hour.

7. Illustration of how to Read Device

FIG. 6 provides a schematic view of possible results that may be obtained when using a three channel device according to the invention wherein the device contains:

Channel 9 (first reporting means):

WST-9

mPMS

PVP

Channel 2 (second reporting means):

WST-9

mPMS

PVP

Vancomycin

Channel 3 (leukocyte detection means):

MTT

MES

8. Further Example Device of the Invention

A further example of the invention was prepared comprising the following components in amounts to provide the concentrations specified assuming 16 mL of PD effluent is provided to each channel.

| | | Final |
|---|---|---|
| | | concentration |
| | Substance | mg/mL |
| Channel 1 | Wst-9 | 0.3778 |
| (first reporting means) | mPMS | 0.008415 |
| | Wilkins Chalgren Broth | 11.6 |
| | Sodium bicarbonate | 0.31 |
| | Digitonin | 0.08 |
| | PVP | >0.8 |
| Channel 2 | Wst-9 | 0.3778 |
| (second reporting means) | mPMS | 0.008415 |
| | Wilkins Chalgren Broth | 11.6 |
| | Sodium bicarbonate | 0.31 |
| | Digitonin | 0.08 |
| | Vancomycin | 0.016 |
| | PVP | >0.8 |
| Channel 3 | Wst-9 | 0.3778 |
| (leukocyte detection means) | mPMS | 0.0168 |
| | MES hydrate | 4.881 |
| | Sodium bicarbonate | |
| | Meropenem | 0.016 |
| | Ciprofloxacin | 0.008 |
| | Vancomycin | 0.008 |
| | PVP | >2.5 |

FIG. 7 shows photographs of channels of the above device (example 8) when exposed to a variety of clinical samples. In this figure when we say that bacteria or leukocyte cells are present or not present we mean that they are present above the threshold concentration or not present above the threshold concentration.

1. A method of determining the presence of a threshold concentration of leukocyte cells in a sample, the method comprising (i) contacting the sample with a leukocyte detection means comprising (a1) an indicator compound; and (ii) examining the leukocyte detection means.

2. A method according to claim 1 wherein the sample is selected from peritoneal dialysis effluent and fluid drained from the peritoneal cavity present due to ascites.

3. A method according to claim 1 wherein the sample is peritoneal dialysis effluent.

4. A method according to 1, wherein the indicator compound (a1) is a tetrazolium compound.

5. A method according to 1, which further involves analysing the sample for the presence of microorganisms wherein step (i) further comprises contacting the sample with:
(II) a first reporting means comprising:
(a2) an indicator compound; and
(d1) media and/or nutrients that support or encourage microbial growth.

6. A method according to claim 1, wherein step (i) further comprises contacting the sample with (III) a second reporting means wherein the second reporting means comprises:
(a3) an indicator compound;
(d2) media and/or nutrients that support or encourage microbial growth;
and
(e) a selection factor.

7. A method according to claim 5, wherein the indicator compound (a2) in the first reporting means is a water soluble tetrazolium salt.

8. A method according to claim 5 wherein the media and/or nutrients that support or encourage microbial growth (d1) in the first reporting means is Wilkins Chalgren media.

9. A method according to claim 5 wherein the first reporting means further comprises an electron mediator.

10. A method according to claim 1, wherein the leukocyte detection means further comprises an electron mediator.

11. A method according to claim 10 wherein the electron mediator is 1-Methoxy-5-methylphenazinium methylsulfate (mPMS).

12. A method according to claim 6, wherein the selection factor inhibits the growth of Gram positive microorganisms.

13. A method according to claim 12 wherein the selection factor is vancomycin.

14. A method according to 1, wherein the leukocyte detection means further comprises one or more antibacterial agents.

15. A method according to claim 5, wherein the first reporting means reporting means further comprises a leukocyte inhibiting agent.

16. A method according to 1, wherein step (ii) involves examining the leukocyte detection means.

17. A device for detecting a threshold concentration of leukocytes in a sample, the device comprising a channel arranged to receive the sample wherein the sample comprises (a 1) an indicator compound and a buffer.

18. A device according to claim 17 which is also useful for detecting and/or identifying microorganisms that may be present in the sample, said device comprising three channels that are arranged to receive the fluid and wherein:
a first channel contains a first reporting means comprising:
(a2) an indicator compound; and
(d1) media and/or nutrients that support or encourage microbial growth;
and
a second channel contains a second reporting means comprising:
(a3) an indicator compound;
(d2) media and/or nutrients that support or encourage microbial growth;
and
(c) a selection factor which selectively inhibits growth of microorganisms;
and
a third channel contains a leukocyte detection means comprising:
(a 1) an indicator compound; and
(b) a buffer.

19. A device according to claim 18 wherein each of the first and second channels further comprises an electron mediator and the third channel further comprises a buffer.

20. The device according to claim 18 wherein the channels are retained within a casing and said casing has viewing windows for observing the contents of the channels.
